(19) United States
(12) Patent Application Publication
(10) Pub. No.: US 2010/0190694 A1
(43) Pub. Date: Jul. 29, 2010

(54) Methods for identifying patients who will respond well to cancer treatment

(76) Inventor: Jan Fagerberg, Smedstorp (SE)

Correspondence Address: KLAUBER & JACKSON, 411 HACKENSACK AVENUE, HACKENSACK, NJ, 07601, US

(21) Appl. No.: 12/657,160

(22) Filed: Jan. 14, 2010

Related U.S. Application Data
(60) Provisional application No. 61/205,124, filed on Jan. 14, 2009.

(30) Foreign Application Priority Data
Jan. 14, 2009 (GB) GB0900555.4

(51) Int. Cl. A61K 31/519; A61K 31/513; A61K 31/7068; A61K 31/517; A61K 38/15; A61P 35/00; C12Q 1/68

(52) U.S. Cl. 514/11; 514/274; 514/49; 514/265.1; 514/249; 514/266.24; 435/6

(57) ABSTRACT

The invention provides methods for identifying patients who will respond well to cancer treatment with a therapeutic regimen that comprises the use of a histone deacetylase inhibitor (HDACi) and one or more further chemotherapeutic agents. The invention also relates to methods of treating such patients with a therapeutic regimen comprising the use of a histone deacetylase inhibitor (HDACi) and one or more further chemotherapeutic agents.

### CROSS-REFERENCE TO RELATED APPLICATIONS

The present application is a non-provisional application claiming the priority of copending provisional application Ser. No. 61/205,124, filed Jan. 14, 2009, and of GB Application 09000555.4, filed Jan. 14, 2009, the disclosures of which are incorporated by reference herein in their entirety. Applicants claim the benefits of these applications under 35 U.S.C. §119.

### FIELD OF THE INVENTION

The present invention relates to methods for identifying patients who will respond well to cancer treatment with a therapeutic regimen that comprises the use of a histone deacetylase inhibitor (HDACi) and one or more further chemotherapeutic agents. The invention also relates to methods of treating such patients with a therapeutic regimen comprising the use of a histone deacetylase inhibitor (HDACi) and one or more further chemotherapeutic agents.

### BACKGROUND

Histones are major protein components of chromatin. The regulation of chromatin structure is emerging as a central mechanism for the control of gene expression. As a general paradigm, acetylation of the ε-amino groups of lysine residues in the amino-terminal tails of nucleosomal histones is associated with transcriptional activation, while deacetylation is associated condensation of chromatin and transcriptional repression. Acetylation and deacetylation of histones is controlled by the enzymatic activity of histone acetyltransferases (HATs) and histone deacetylases (HDACs). Several transcription factors including p53 and GATA-1 have also been shown to be substrates for HDACs.

Prototypical HDAC inhibitors, such as the natural products trichostatin A (TSA) and suberoyl hydroxamic acid (SAHA), induce the expression of genes associated with cell cycle arrest and tumour suppression. Phenotypic changes induced by HDAC inhibitors (HDACi) include G1, and G2/M cell cycle arrest, induction of apoptosis in tumour cells, inhibition of angiogenesis, immune modulation and promotion of cellular differentiation. HDACi also modulate gene expression within tumour cells, including tumour suppressor genes. Antitumour activity has been demonstrated in vivo in animal models with a number of HDAC inhibitors, including PXD-101 (also known as Belinostat).

PXD-101 is a potent HDAC inhibitor that belongs to the hydroxymate-type of histone deacetylase inhibitors, which for various members of the group have shown pronounced in vitro and in vivo (pre-clinical and early clinical trials) activity against lymphoma.

HDAC inhibitors, such as PXD-101 which is a small molecule class I and class II HDAC inhibitor, have found use in the treatment of cancer.

HDACi have been shown to act synergistically with further chemotherapeutic agents. Thus, WO 2006/082428 describes methods for the treatment of cancer comprising administering to a patient a first amount or a dose of an HDACi and a second amount or dose of another chemotherapeutic agent or an epidermal growth factor receptor inhibitor such as Tarceva®. The second amount or dose is of a compound selected from Cisplatin, 5-Fluorouracil, Oxaliplatin, Topotecan, Gemcitabine, Docetaxel, Doxorubicin, Tamoxifen, Dexamethasone, 5-Azacytidine, Chlorambucil, Fludarabine, Tarceva®, Alimta®, Melphalan, and pharmaceutically acceptable salts and solvates thereof.

WO 2007/054719 describes a method for the treatment of a haematological cancer comprising admininstering to a patient an HDACi as well as a method of treating a solid tumour cancer comprising admininstering a first amount of an HDACi and a second amount of another chemotherapeutic agent selected from an antibody against VEGF, Avastin®, an antibody against CD20, rituximab, bortezomib, thalidomide, dexamethasone, vincristine, doxorubicin, and melphalan (also known as L-PAM and PAM).

PXD-101 has been shown in vitro to reduce the expression of thymidylate synthase (TS) in HCT116 colon cancer cells (Tumber et al., Cancer Chemother. Pharmacol. (2007) 60:275-283) and it is postulated that this provides a mechanistic rationale for the synergy demonstrated between PXD-101 and 5-fluorouracil, as demonstrated in WO 2006/082428.

Elevated levels of TS have been demonstrated in colon cancer cells with both de novo and acquired resistance to fluoropyrimidines, and low intratumoral TS levels have been shown to predict both improved response and survival in patients with metastatic colorectal cancer who were treated with fluoropyrimidine-based chemotherapy (Aschele JCO 1999, Leichman JCO 1997). In addition, it has been demonstrate that colorectal tumours responding to 5-fluorouracil have low gene expression levels of TS, and in addition low levels of thymidine phosphorylase (TP) and dihydropyrimidine dehydrogenase (DPD) (Salonga et al., Clinical Cancer Research 6:1322-1327, 2000).

### SUMMARY OF THE INVENTION

The present inventors have surprisingly found that the clinical outcome in cancer patients treated with a therapeutic regimen comprising the use of a histone deacetylase inhibitor (HDACi) and one or more further chemotherapeutic agents can be predicted based on the change in the expression patterns of three genes, thymidylate synthase (TS), dihydropyrimidine dehydrogenase (DPD) and p21, in response to a test dose of an HDACi.

In particular, the present inventors have found that patients showing at least two of the following: a decrease in the expression of TS, a decrease in the expression of DPD, and an increase in expression of p21, are likely to have a favourable clinical outcome. This is also referred to as a “2 out of 3” expression pattern in the application.

Specifically, patients with a “2 out of 3” expression pattern showed a longer period of stabilization of the disease compared to patients who did not show such an expression pattern in response to a test dose of an HDACi. In addition, these patients showed either a longer, or an approximately equal, period of stabilization of the disease compared to the period of stabilization observed in response to the most recent prior line of therapy they received. In contrast, patients who did not show this pattern of expression did not have a favourable clinical outcome and showed a shorter period of stabilization of the disease compared to the period of stabilization observed in response to their most recent prior line of therapy.

Accordingly, in one aspect there is provided a method of assessing the susceptibility of a subject to cancer treatment with a therapeutic regimen comprising the use of a histone deacetylase inhibitor (HDACi) and one or more further chemotherapeutic agents, the method comprising:

determining the level of expression of TS, DPD and p21 after administration of an initial dose of HDACi,

wherein a subject susceptible to treatment with the therapeutic regimen comprising the use of a histone deacetylase inhibitor (HDACi) and one or more further chemotherapeutic agents shows at least two of: a decrease in expression of TS, a decrease in expression of DPD, and an increase in expression of p21, after administration of the initial dose of the HDACi.

In another aspect there is provided a method of assessing the susceptibility of a subject to cancer treatment with a therapeutic regimen comprising the use of a histone deacetylase inhibitor (HDACi) and one or more further chemotherapeutic agents, the method comprising:

(i) administering an initial dose of an HDACi to the subject or to a sample isolated from the subject;

(ii) determining the level of expression of TS, DPD and p21; and

(iii) comparing the level of expression of TS, DPD and p21 before and after administration of the initial dose of the HDACi,

wherein a subject susceptible to treatment with the therapeutic regimen comprising the use of a histone deacetylase inhibitor (HDACi) and one or more further chemotherapeutic agents, shows at least two of: a decrease in expression of TS, a decrease in expression of DPD, and an increase in expression of p21, after administration of the initial dose of the HDACi.

In another aspect there is provided a method of treating cancer in a subject, the method comprising:

(i) administering an initial dose of a histone deacetylase inhibitor (HDACi) to the subject or to a sample isolated from the subject;

(ii) determining the level of expression of TS, DPD and p21;

(iii) comparing the level of expression of TS, DPD and p21 before and after administration of the initial dose of the HDACi; and

(iv) administering a therapeutically effective amount of a therapeutic regimen comprising an HDACi and one or more further chemotherapeutic agents to the subject, provided that the subject showed at least two of: a decrease in expression of TS, a decrease in expression of DPD, and an increase in expression of p21 after administration of the initial dose of the HDACi.

In another aspect there is provided a histone deacetylase inhibitor (HDACi) for use in a method of treating cancer, the method comprising administering a therapeutic regimen comprising the use of an HDACi and one or more further chemotherapeutic agents to an subject in need thereof, wherein the subject is selected by showing at least two of: a decrease in the expression of TS, a decrease in expression of DPD, and an increase in expression of p21 in response to administration of an initial dose of an HDACi.

In another aspect there is provided one or more chemotherapeutic agents for use in a method of treating cancer, the method comprising administering a therapeutic regimen comprising the use of the chemotherapeutic agent(s) and a histone deacetylase inhibitor (HDACi) to an subject in need thereof, wherein the subject is selected by showing at least two of: a decrease in the expression of TS, a decrease in expression of DPD, and an increase in expression of p21 in response to administration of an initial dose of the HDACi.

In another aspect there is provided the use of a histone deacetylase inhibitor (HDACi) in the manufacture of a medicament for the treatment of cancer in a subject, wherein the treatment comprises administering a therapeutic regimen comprising the HDACi and one or more further chemotherapeutic agents to the subject and wherein the subject is selected by showing at least two of: a decrease in the expression of TS, a decrease in expression of DPD, and an increase in expression of p21 after administration of an initial dose of the HDACi.

In a further aspect there is provided the use of one or more chemotherapeutic agents in the manufacture of a medicament for the treatment of cancer in a subject, wherein the treatment comprises administering a therapeutic regimen comprising the chemotherapeutic agent(s) and a histone deacetylase inhibitor (HDACi) to the subject wherein the subject is selected by showing at least two of: a decrease in the expression of TS, a decrease in expression of DPD, and an increase in expression of p21 after administration of an initial dose of the HDACi.

These and further aspects of the invention are set out in more detail below.

### BRIEF DESCRIPTION OF THE DRAWINGS

FIG. 1 depicts a graph illustrating the increase in progression-free survival (PFS) in patients showing a “2 out of 3” marker pattern (n=6; solid line) compared with patients not showing a “2 out of 3” marker pattern (n=14; dashed line). Progression free survival between the two patient groups differed significantly (p<0.04).

FIG. 2 depicts a graph which compares progression free survival of patients having a “2 out of 3” marker pattern and treated with PXD-101/5-FU (triangles) compared with progression free survival of the same patients in response to their most recent previous treatment (squares).

FIG. 3 depicts a graph which compares progression free survival of patients not demonstrating a “2 out of 3” marker pattern and treated with PXD-101/5-FU (diamonds) compared with progression free survival of the same patients in response to their most recent previous treatment (squares).

FIG. 4 Treatment cycles 21 days included Bel (PXD-101) alone in cycle 1, and Bel in combination with FU in all subsequent cycles

### DETAILED DESCRIPTION OF THE INVENTION

Although cancer treatment as described herein requires the use of an HDAC inhibitor and one or more further chemotherapeutic agents, the treatment may also include additional therapeutic, non-therapeutic or chemotherapeutic agents as described herein.

Reference to a therapeutic regimen comprising the use of an HDACi and one or more chemotherapeutic agents as used herein includes a regimen consisting of the use of an HDACi and one or more chemotherapeutic agents, as well as a regimen which comprises the use of an HDACi, one or more chemotherapeutic agents and one or more additional therapeutic or non-therapeutic agents, as described herein.

As used herein, reference to treatment includes any treatment for the killing or inhibition of growth of a tumour cell. This includes treatment intended to alleviate the severity of a tumour, such as treatment intended to cure the tumour or to provide relief from the symptoms associated with the tumour. It also includes prophylactic treatment directed at preventing or arresting the development of the tumour in an individual at risk from developing a tumour. For example, the treatment may be directed to the killing of micro-metastases before they become too large to detect by conventional means.

The therapeutic agents or the therapeutic regimen defined herein may be administered simultaneously, separately or sequentially. By “simultaneous” administration, it is meant that the HDACi and further chemotherapeutic agent are administered to the subject in a single dose by the same route of administration.

By “separate” administration it is meant that the HDACi and further chemotherapeutic agent are administered to the subject by two different routes of administration which occur at the same time. This may occur for example when one component is administered by infusion and the other is given orally during the course of the infusion.

By “sequential” administration it is meant that the HDACi and further chemotherapeutic agent are administered at different points in time, provided that the activity of the first administered agent is present and ongoing in the subject at the time the second agent is administered. It may be that the HDACi is the first agent to be administered and the further chemotherapeutic agent is the second agent to be administered, or vice versa.

The term “subject” or “patient” as used herein is intended to mean a mammalian or a non-mammalian subject. In one embodiment, the subject is a mammal, such as a human, canine, murine, feline, bovine, ovine, swine, or caprine. In a preferred embodiment, the subject is a human.

In the methods of the present invention, the level of expression of TS, DPD and p21 may be determined after exposure of the subject or a sample therefrom to an initial dose of the HDACi.

Thymidylate synthase (TS) is the enzyme used to generate thymidine monophosphate (dTMP), which is subsequently phosphorylated to thymidine triphosphate for use in DNA synthesis and repair. TS is a target for multiple chemotherapeutics and has been the focus of many studies into the efficacy of chemotherapeutics. For example, Salonga et al., Clinical Cancer Research 6:1322-1327 (2000) show that colorectal cancers that respond to the chemotherapeutic 5-fluorouracil have low gene expression levels of TS, as well as of dihydropyrimidine dehydrogenase and thymidine phosphorylase. Elevated levels of TS have also been demonstrated in colon cancer cells with both de novo and acquired resistance to fluoropyrimidines, and low intratumoral TS levels have been shown to predict both improved response and survival in patients with metastatic colorectal cancer who were treated with fluoropyrimidine-based chemotherapy (Aschele JCO 1999, Leichman JCO 1997).

Dihydropyrimidine dehydrogenase (DPD) is an enzyme that is involved in pyrimidine degradation. It is the initial and rate-limiting step in pyrimidine catabolism. It catalyzes the reduction of uracil and thymine. Studies over the past two decades have demonstrated that DPD is an important regulatory enzyme in the metabolism of both the naturally occurring pyrimidines uracil and thymine as well as the cancer chemotherapy fluoropyrimidine drug, 5-FU (Diasio, R. B. The role of dihydropyrimidine dehydrogenase (DPD) modulation in 5-FU pharmacology. Oncology, 12 (10 Suppl. 7): 23-27, 1998) and other fluoropyrimidine drugs such as capecitabine (Xeloda) and others. In particular, pharmacokinetic studies have demonstrated that 85% of clinically administered 5-FU is inactivated and eliminated through the catabolic pathway (Heggie, G. C., Sommadossi, J. P., Cross, D. S., Huster, W. J., and Diasio, R. B. Clinical pharmacokinetics of 5-fluorouracil and its metabolites in plasma, urine, and bile. Cancer Res., 47: 2203-2206, 1987). However, the cytotoxicity of 5-FU in host and tumour cells only occurs following anabolism to nucleotides with the amount of 5-FU available for anabolism being determined by the extent of its catabolism (Grem, J. L. Fluoropyrimidines. In: B. A. Chabner and D. L. Longo (eds.), Cancer Chemotherapy and Biotherapy, Ed. 2, pp. 149-197. Philadelphia: Lippincott-Raven, 1996). Thus, a balance exists between the enzymatic activation of 5-FU and its catabolic elimination with the DPD enzyme being recognized as an essential factor in the overall regulation of 5-FU metabolism.

p21 is the gene encoding cyclin dependent kinase inhibitor 1A, which is also known as Cipl and CDKNIA. The encoded protein binds to and inhibits the activity of cyclin-CDK2 or -CDK4 complexes, and thus functions as a regulator of cell cycle progression at G1. The expression of this gene is tightly controlled by the tumor suppressor protein p53, through which this protein mediates the p53-dependent cell cycle G1 phase arrest in response to a variety of stress stimuli. This protein can interact with proliferating cell nuclear antigen (PCNA), a DNA polymerase accessory factor, and plays a regulatory role in S phase DNA replication and DNA damage repair. This protein was reported to be specifically cleaved by CASP3-like caspases, which thus leads to a dramatic activation of CDK2, and may be instrumental in the execution of apoptosis following caspase activation.

In this invention, the susceptibility of a subject to cancer treatment with a therapeutic regimen comprising an HDACi and one or more further chemotherapeutic agents is assessed by determining the levels of expression of each of TS, DPD and p21 after exposure of the patient or a sample isolated therefrom to an initial dose of an HDACi.

The present inventors have discovered that the demonstration of a specific pattern in the levels of expression of the markers TS, DPD and p21 caused by the initial dose of an HDACi, allows a prediction of an increased progression free survival after treatment with the therapeutic agents defined herein.

In a preferred embodiment of the present invention, patients who demonstrate two or more of: a decrease in the expression of TS, a decrease in the expression of DPD and an increase in the expression of p21 following an initial dose of an HDACi, will show an increased progression free survival after treatment with the therapeutic agents defined herein.

The increase or decrease in the expression of TS, DPD and/or p21 is decided relative to a baseline measurement of the expression of these markers, wherein the baseline measurement is taken prior to the exposure of the patient to the initial dose of an HDACi.

In a preferred embodiment, therefore, the present invention provides a method of assessing the susceptibility of a subject to cancer treatment with a therapeutic regimen comprising the use of a histone deacetylase inhibitor (HDACi) and one or more further chemotherapeutic agents, the method comprising:

(i) determining the level of expression of TS, DPD and p21 in a sample isolated from a subject prior to administration of an HDACi;

(ii) determining the level of expression of TS, DPD and p21 after administration of an initial dose of HDACi,

(iii) comparing the expression levels of TS, DPD and p21 before and after administration of an HDACi;

wherein a subject susceptible to treatment with the therapeutic regimen comprising the use of a histone deacetylase inhibitor (HDACi) and one or more further chemotherapeutic agents shows at least two of: a decrease in expression of TS, a decrease in expression of DPD, and an increase in expression of p21, after administration of the initial dose of the HDACi.

In an alternative preferred embodiment there is provided a method of assessing the susceptibility of a subject to cancer treatment with a therapeutic regimen comprising the use of a histone deacetylase inhibitor (HDACi) and one or more further chemotherapeutic agents, the method comprising:

(i) determining the level of expression of TS, DPD and p21 in a sample from a subject;

(ii) administering an initial dose of an HDACi to the subject or to a sample isolated from the subject;

(iii) determining the level of expression of TS, DPD and p21; and

(iv) comparing the level of expression of TS, DPD and p21 before and after administration of the initial dose of the HDACi,

wherein a subject susceptible to treatment with the therapeutic regimen comprising the use of a histone deacetylase inhibitor (HDACi) and one or more further chemotherapeutic agents, shows at least two of: a decrease in expression of TS, a decrease in expression of DPD, and an increase in expression of p21, after administration of the initial dose of the HDACi.

In a further alternative preferred embodiment there is provided a method of treating cancer in a subject, the method comprising:

(i) determining the level of expression of TS, DPD and p21 in a sample from a subject;

(ii) administering an initial dose of a histone deacetylase inhibitor (HDACi) to the subject or to a sample isolated from the subject;

(ii) determining the level of expression of TS, DPD and p21;

(iv) comparing the level of expression of TS, DPD and p21 before and after administration of the initial dose of the HDACi; and

(v) administering a therapeutically effective amount of a therapeutic regimen comprising an HDACi and one or more further chemotherapeutic agents to the subject,

provided that the subject showed at least two of: a decrease in expression of TS, a decrease in expression of DPD, and an increase in expression of p21 after administration of the initial dose of the HDACi.

A determination of the levels of expression of each of TS, DPD and p21 can be made using any appropriate technique. In a preferred embodiment, this determination is made using RTQ-PCR (quantitative real time PCR). RTQ-PCR allows amplification and simultaneous quantification of a target DNA molecule. To analyze gene expression levels using quantitative PCR, the total mRNA of a cell is first isolated and reverse transcribed into DNA using reverse transcriptase. For example, mRNA levels can be determined using a Taqman Gene Expression Assays (Applied Biosystems) on an ABI PRISM 7900HT instrument according to the manufacturer's instructions. Transcript abundance can then be calculated by comparison to a standard curve.

The expression levels of each of TS, DPD and p21 can be determined in any appropriate sample obtained from a subject. In preferred embodiments, the sample is a peripheral blood mononuclear cell (PBMC) sample, a mucosal sample or a tumour sample.

It is generally preferred that the sample used to determine the baseline expression levels of TS, DPD and p21 and the sample used to determine the levels of expression of these markers after administration of an HDACi are of the same type, i.e. both samples are PBMC or both samples are tumour samples, although it is envisaged that the samples may be of different type.

For determination of the baseline expression levels and for determination of expression levels of the markers after administration of an HDACi, the level of expression of TS, DPD and p21 may be determined in each case in a single sample obtained from the subject. Preferably however, two or more samples obtained from the subject are analysed to determine expression levels of these markers both for the baseline determination and after administration of an HDACi. Most preferably, the expression of TS, DPD and p21 is determined, both in the case of the baseline determination and the determination of expression levels after administration of an HDACi, in two samples obtained from the subject. For either or both of the determination of the baseline levels of expression of the markers and the determination of the levels of these markers after exposure to the HDACi, when the expression levels of the markers are determined in more than one sample, the level of expression of TS, DPD and p21 determined may the mean of the level of expression measured in the samples. This may improve the accuracy of the determination.

In this invention, a determination of the levels of expression of the markers TS, DPD and p21 is made after exposure of the patient or of a sample isolated therefrom to an initial dose of the HDACi, and the levels of expression compared to the levels of expression before exposure to the initial dose of the HDACi.

In the methods of this invention, administration of an initial dose of an HDACi may be either to a patient or to a sample obtained therefrom. Thus, the present invention includes the situation where a sample is obtained from a subject and that sample or a part thereof is used for determination of a baseline level of expression of TS, DPD and p21 and then that sample or a part thereof is exposed to an initial dose of an HDACi, followed by a determination of the level of expression of TS, DPD and p21 post HDACi exposure. In other words, the level of expression of the markers TS, DPD and p21 may be determined in vitro. In an alternative situation, the patient is treated directly with an initial dose of an HDACi, such that the baseline determination of the expression levels of TS, DPD and p21 and the determination of the levels of expression of these markers post said HDACi treatment are made on samples isolated from the patient at the appropriate time.

If the patient is treated directly with an initial dose of an HDACi, the sample for determining the expression of TS, DPD and p21 after administration of the initial dose of the HDACi may be taken at any suitable time point after the administration of said initial dose of the HDACi. By any suitable time point is meant at any time point after the HDACi has had sufficient time to exert its effect. For example, the sample may be taken between 6 and 24 hours after administration of the initial dose of the HDACi. Preferably, the sample is taken 6 hours after the administration of the initial dose of the HDACi.

In the methods of the present invention, the initial dose of an HDACi is typically a standard dose of the HDACi, i.e. that amount of an HDACi that a clinician would use during a treatment involving the use of an HDACi. The initial dose of an HDACi may be administered in the same manner as would a therapeutic dose of an HDACi and the amounts of the dose might differ depending on the mode of administration. Thus, if the initial dose is administered orally, the amount can be between about 2 mg to about 6000 mg per day, such as from about 20 mg to about 6000 mg per day, such as from about 200 mg to about 6000 mg per day. If the initial dose is administered intravenously or subcutaneously, the subject would receive the HDAC inhibitor in quantities sufficient to deliver between about 3-1500 mg/m² per day, for example, about 3, 30, 60, 90, 180, 300, 600, 900, 1000, 1200, or 1500 mg/m² per day.

In the methods of the present invention, the initial dose of an HDACi may be administered as a single dose on a single day or as a single dose over a period of days, e.g. daily over a period of between 1 and 5 days.

In the methods of the present invention, the initial dose of an HDACi may be administered to a sample in vitro. Preferably, the initial dose administered to the sample in this case is chosen such that the concentration of the HDACi in the sample reflects the concentration of the HDACi used for treatment.

In this invention, the determination of the levels of expression of each of TS, DPD and p21 will allow an inference as to whether or not a patient is likely to have a positive clinical response to the therapeutic compounds defined herein. A positive clinical response may for example be a long stabilization of the disease, i.e. a longer time to (disease) progression [TTP]. Other positive clinical responses include tumour shrinkage and prolonged patient survival.

In a preferred embodiment, a patient will be determined to be suitable for treatment with the therapeutic compounds defined herein if the expression levels of two of the three markers, TS, DPD and p21, have been determined to differ from the baseline measurements.

In one embodiment, a patient will be determined to be suitable for treatment with the therapeutic compounds defined herein if the expression levels of TS and DPD have been determined to differ from the baseline measurements.

In one embodiment, a patient will be determined to be suitable for treatment with the therapeutic compounds defined herein if the expression levels of TS and p21 have been determined to differ from the baseline measurements.

In one embodiment, a patient will be determined to be suitable for treatment with the therapeutic compounds defined herein if the expression levels of DPD and p21 have been determined to differ from the baseline measurements.

More preferably, a patient will be determined to be suitable for treatment with the therapeutic compounds defined herein if the expression levels of all of the three markers, TS, DPD and p21, have been determined to differ from the baseline measurements.

Most preferably, a patient will be determined to be suitable for treatment with the therapeutic compounds defined herein if the expression levels of all three markers, TS, DPD and p21, have been determined to differ from the baseline measurements, and the expression levels show a decrease in the expression of TS, a decrease in expression of DPD, and an increase in expression of p21 compared to the baseline measurements.

In one aspect, the methods of the invention allow the identification of a patient with cancer most likely to benefit from treatment with the therapeutic agents defined herein.

As used herein, the term “cancer” refers to tumors, neoplasms, carcinomas, sarcomas, leukemias, lymphomas, and the like. For example, cancers include, but are not limited to, leukemias and lymphomas such as cutaneous T-cell lymphoma (CTCL), noncutaneous peripheral T-cell lymphoma, lymphomas associated with human T-cell lymphotropic virus (HTLV), for example, adult T-cell leukemia/lymphoma (ATLL), acute lymphocytic leukemia, acute nonlymphocytic leukemias, chronic lymphocytic leukemia, chronic myelogenous leukemia, Hodgkin's Disease, non-Hodgkin's lymphomas, and multiple myeloma, solid tumors such as brain tumors, neuroblastoma, retinoblastoma, Wilms' Tumor, bone tumors, and soft-tissue sarcomas, common solid tumors such as head and neck cancers (e.g., oral, laryngeal and esophageal), genitourinary cancers (e.g., prostate, bladder, renal, uterine, ovarian, testicular, rectal and colon [colorectal]), lung cancer, non-small cell lung cancer, prostate cancer, breast cancer, pancreatic cancer, melanoma and other skin cancers, stomach cancer, brain cancer, liver cancer, thyroid cancer, and thymic malignancies (including epithelial mediastinal thymoma), and gastric cancer.

In a preferred embodiment, the cancer to be treated is selected from the group of: colorectal cancer, pancreatic cancer, esophagal cancer, prostate cancer, non small cell lung cancer, gastric cancer, head and neck cancer, non-Hodgkin lymphoma and breast cancer.

In the context of the methods of treatment described herein which refer to two active agents (e.g., an HDAC inhibitor and a further chemotherapeutic agent, as described herein), the term “therapeutically effective amount” is intended to qualify the combined amount of the first and second agents in the therapy. The combined amount will achieve the desired biological response, for example, partial or total inhibition, delay or prevention of the progression of cancer including cancer metastasis; inhibition, delay or prevention of the recurrence of cancer including cancer metastasis; or the prevention of the onset or development of cancer (chemoprevention) in a mammal, for example a human.

The methods of the invention may be applicable to any subject suffering from cancer and requiring treatment, regardless of whether the cancer is newly diagnosed or the patient is undergoing cancer treatment of any form. The methods of the invention can also be applied to any subject who is undergoing adjuvant or neo-adjuvant treatment.

### Histone Deacetylase Inhibitors

Histone deacetylases are involved in the reversible acetylation of histone and non-histone proteins (p53, tubulin, and various transcription factors). Mammalian HDACs have been ordered into three classes based upon their similarity to known yeast factors. Class I HDACs (HDACs 1, 2, 3 and 8) bear similarity to the yeast RPD3 protein, are located in the nucleus and are found in complexes associated with transcriptional co-repressors. Class II HDACs (HDACs 4, 5, 6, 7 and 9) are similar to the yeast HDA1 protein, and have both nuclear and cytoplasmic subcellular localization. Class III HDACs form a structurally distant class of NAD dependent enzymes that are related to the yeast SIR2 proteins.

Compounds that are shown to inhibit HDAC activity fall into five structurally diverse classes: (1) hydroxamic acids; (2) cyclic tetrapeptides; (3) aliphatic acids; (4) benzamides; and (5) electrophilic ketones.

Hydroxamic acids were among the first HDAC inhibitors identified and these agents helped define the model pharmacophore for HDAC inhibitors. The linker domain of these agents is comprised of linear or cyclic structures, either saturated or unsaturated, and the surface recognition domain is generally a hydrophobic group, most often aromatic. Phase I and II clinical trials are currently on-going for several hydroxamic acid based HDAC inhibitors, including PXD-101.

PXD-101 is a highly potent HDAC inhibitor that blocks proliferation of diverse tumour cell lines at low micromolar potency (IC₅₀ 0.08-2.43 μM) and HDAC enzyme activity (IC₅₀ 9-110 nM). In xenograft models, PXD-101 slows tumour growth. In addition, PXD-101 causes cell cycle arrest and apoptosis in rapidly proliferating cells.

Hydroxamic acid based HDAC inhibitors are particularly suitable for use in the present invention.

In one embodiment, the HDAC inhibitor used in the present invention is selected from compounds of the following formula and pharmaceutically acceptable salts and solvates thereof:

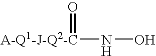

wherein:

A is an unsubstituted phenyl group;

Q¹ is a covalent bond, a C₁₋₇alkylene group, or a C₂₋₇alkenylene group;

J is:

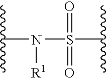

R¹ is hydrogen, C₁₋₇alkyl, C₃₋₂₀heterocyclyl, C₅₋₂₀aryl, or C₅₋₂₀aryl-C₁₋₇alkyl; and,

Q² is:

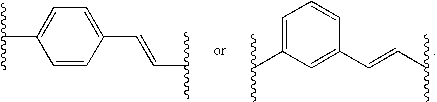

In one embodiment, Q¹ is a covalent bond, a C₁₋₄alkylene group, or a C₂₋₄alkenylene group.

In one embodiment, Q¹ is a covalent bond.

In one embodiment, Q¹ is a C₁₋₇alkylene group.

In one embodiment, Q1 is —CH2-, —C(CH3)-, —CH2CH2-, —CH2CH(CH3)-, —CH2CH(CH2CH3)-, —CH₂CH₂CH₂—, or —CH₂CH₂CH₂CH₂—.

In one embodiment, Q¹ is a C₂₋₇alkenylene group.

In one embodiment, Q¹ is —CH═CH— or —CH═CH—CH₂—.

In one embodiment, R¹ is hydrogen or C₁₋₇alkyl.

In one embodiment, R¹ is hydrogen or C₁₋₃alkyl.

In one embodiment, R¹ is hydrogen, -Me, -Et, -nPr, -iPr, -nBu, -iBu, -sBu, or -tBu.

In one embodiment, R¹ is hydrogen, -Me, or -Et.

In one embodiment, R¹ is hydrogen.

In one embodiment, Q² is:

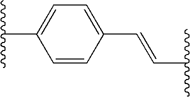

In one embodiment, Q² is:

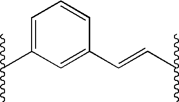

All compatible combinations of the above embodiments are disclosed herein, as if each particular combination was individually and explicitly recited.

In one embodiment, the HDAC inhibitor used in the present invention is selected from the following compounds, and pharmaceutically acceptable salts or solvates thereof:

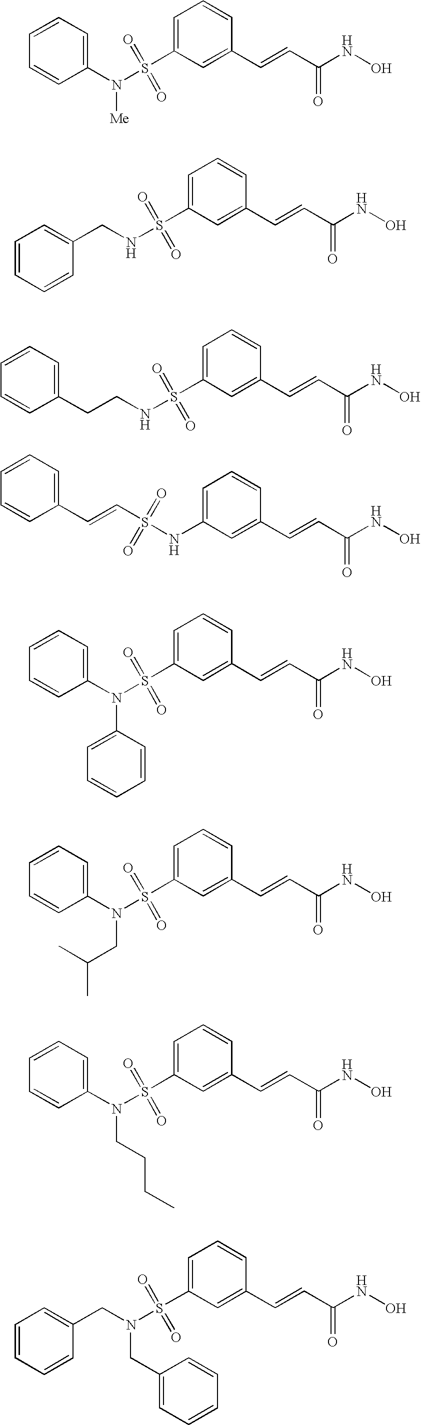

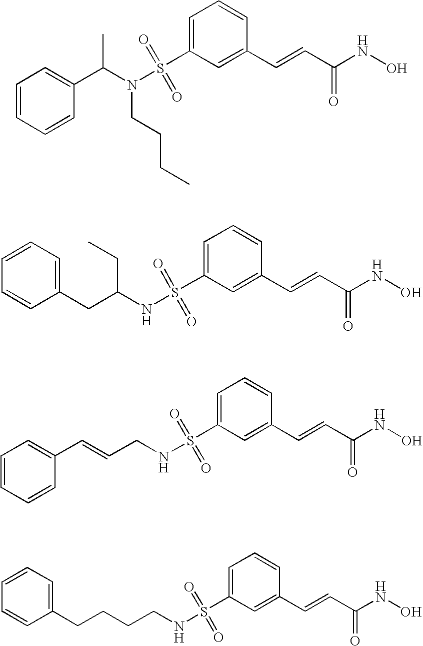

In one embodiment, the HDAC inhibitor used in the present invention is selected from vorinostat, panobinostat (hydroxamate), romidepsin (depsipeptide), SNDX-275, MGCD-0103, PCI24781, CHR-3996, ITF2357, SB939, JNJ26481585, JNJ16241199, valproic acid and the following compound (also known as PXD-101) and pharmaceutically acceptable salts and solvates thereof:

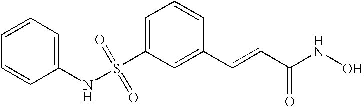

In a preferred embodiment, the HDAC inhibitor used in the present invention is PXD-101.

Other HDAC inhibitors that are suitable for use in the present invention include the compounds disclosed in U.S. Ser. No. 10/381,790; 10/381,794; 10/381,791.

### Stereoisomers

Stereoisomers of the above identified compounds are within the scope of the present invention. Many organic compounds exist in optically active forms having the ability to rotate the plane of plane-polarized light. In describing an optically active compound, the prefixes D and L or R and S are used to denote the absolute configuration of the molecule about its chiral centre(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centres and therefore can exist in different enantiomeric forms. If desired, a chiral carbon can be designated with an asterisk (*). When bonds to the chiral carbon are depicted as straight lines in the formulas of the invention, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When an HDAC inhibitor used in the present invention contains one chiral centre, the compound exists in two enantiomeric forms, and in such cases, references to the compound herein relates to both enantiomers and mixtures of enantiomers, such as the specific 50:50 mixture referred to as a racemic mixture. The enantiomers can be resolved by methods known to those skilled in the art, for example by formation of diastereoisomer salts which can be separated, for example, by crystallization (see, e.g., CRC Handbook of Optical Resolutions via Diastereomeric Salt Formation by David Kozma (CRC Press, 2001)); by formation of diastereoisomer derivatives or complexes which can be separated, for example, by crystallization, gas-liquid or liquid chromatography; by selective reaction of one enantiomer with an enantiomer-specific reagent, for example enzymatic esterification; or by gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon is understood to mean that the designated enantiomeric form of the compounds is in enantiomeric excess (ee) or in other words is substantially free from the other enantiomer. For example, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. Enantiomeric excess, as used herein, is the presence of a particular enantiomer at greater than 50%. For example, the enantiomeric excess can be about 60% or more, such as about 70% or more, for example about 80% or more, such as about 90% or more. In a particular embodiment when a specific absolute configuration is designated, the enantiomeric excess of depicted compounds is at least about 90%. In a more particular embodiment, the enantiomeric excess of the compounds is at least about 95%, such as at least about 97.5%, for example, at least 99% enantiomeric excess.

When an HDAC inhibitor used in the present invention contains two or more chiral carbons it can have more than two optical isomers and can exist in diastereoisomeric forms, and in such cases, references to the compound herein relates to each diastereoisomer of such compounds and mixtures thereof. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers which are not mirror-images (e.g., (S,S) and (R,S)) are diastereomers. The diastereoisomer pairs can be separated by methods known to those skilled in the art, for example chromatography or crystallization and the individual enantiomers within each pair can be separated as described above.

### Salts and Solvates

The active compounds disclosed herein can, as noted above, be prepared in the form of their pharmaceutically acceptable salts. Pharmaceutically acceptable salts are salts that retain the desired biological activity of the parent compound and do not impart undesired toxicological effects. Examples of pharmaceutically acceptable salts are discussed in Berge et al., 1977, “Pharmaceutically Acceptable Salts,” J. Pharm. Sci., Vol. 66, pp. 1-19. The active compounds disclosed can, as noted above, be prepared in the form of their solvates. The term “solvate” is used herein in the conventional sense to refer to a complex of solute (e.g., active compound, salt of active compound) and solvent. If the solvent is water, the solvate may be conveniently referred to as a hydrate, for example, a hemihydrate, monohydrate, dihydrate, trihydrate, tetrahydrate, and the like.

### Prodrugs

Pro-drugs of the HDAC inhibitors disclosed herein are also suitable for use in the present invention. A prodrug of any of the compounds can be made using well known pharmacological techniques.

### Isomers, Homologs, and Analogs

Isomers, homologs and analogs of the HDAC inhibitors disclosed herein are also suitable for use in the present invention. In this context, homologs are molecules having substantial structural similarities to the above-described compounds; analogs are molecules having substantial biological similarities regardless of structural similarities; and isomers are compounds that have the same molecular formula, but different structures (e.g., meta, para, or ortho configurations).

### Chemotherapeutic Agents

Chemotherapeutic agents that are suitable for use in the present invention (i.e., as the one or more further chemotherapeutic agents used as part of the therapeutic regimen that also includes the use of an HDAC inhibitor) include chemotherapeutic agents which exert a therapeutic effect by targeting, wholly or in part, TS. Thus, chemotherapeutic agents suitable for use in the present invention may inhibit or interfere with thymidylate synthase activity either directly or indirectly, inhibit or interfere with the thymidylate synthase expression, and/or interfere with thymidylate synthase by some other mechanism.

Although the exact mechanisms of action of the various chemotherapeutic agents are not essential to the present invention, the inventor postulates that the one or more further chemotherapeutic agents will demonstrate increased efficacy in patients demonstrating lower or decreased expression levels of TS and, optionally, DPD. Accordingly, the one or more chemotherapeutic agents suitable for use in the methods of the present invention are such agents, which demonstrate increased efficacy in patients demonstrating low or decreased expression levels of TS and, optionally, DPD.

Such chemotherapeutic compounds include for example:

fluoropyrimidine compounds, e.g. 5-fluorouracil (FU) and capecitabine (marketed as Xeloda™), and pharmaceutically acceptable salts and solvates thereof;

anti-folate compounds, e.g. pemetrexed (MTA; marketed as Alimta™), pralatrexate (PDX), GW1843, Methotrexate (MTX), Edatrexate (EDX), Aminopterin (AMT), PT523, neutrexin (trimetrexate), and pharmaceutically acceptable salts and solvates thereof;

thymidylate synthase (TS) inhibitors, e.g. thymitaq (AG337), plevitrexed (ZD9331), BGC945, pemetrexed, raltitrexed, and pharmaceutically acceptable salts and solvates thereof; and

anti-metabolite compounds, e.g. ftorafur containing compounds (e.g. tegafur, ftorafur (UFT), and S-1).

In one embodiment, the further chemotherapeutic agent is selected from the group of: 5-fluorouracil (FU), capecitabine, pemetrexed (MTA), pralatrexate (PDX), thymitaq (AG337), plevitrexed (ZD9331), BGC945, raltitrexed, GW1843, methotrexate (MTX), edatrexate (EDX), aminopterin (AMT), PT523, UFT, S-1 and neutrexin (trimetrexate), and pharmaceutically acceptable salts and solvates thereof.

In a preferred embodiment, the further chemotherapeutic agent is selected from the group of: 5-fluorouracil (FU), pemetrexed (MTA), pralatrexate (PDX), thymitaq (AG337), plevitrexed (ZD9331), BGC945, raltitrexed, GW1843, capecitabine, UFT and S-1 and pharmaceutically acceptable salts and solvates thereof.

In a preferred embodiment, the further chemotherapeutic agent is 5-fluorouracil, or a pharmaceutically acceptable salt or solvate thereof.

In a preferred embodiment, the further chemotherapeutic agent is pemetrexed, or a pharmaceutically acceptable salt or solvate thereof.

In a preferred embodiment, the further chemotherapeutic agent is pralatrexate, or a pharmaceutically acceptable salt or solvate thereof.

In a preferred embodiment, the further chemotherapeutic agent is capecitabine, or a pharmaceutically acceptable salt or solvate thereof.

In a preferred embodiment, the further chemotherapeutic agent is selected form the group of 5-fluorouracil (FU), a thymidylate synthase (TS) inhibitor, and an anti-folate compound.

### Therapeutic Regimen

A therapeutic regimen as referred to herein preferably comprises the use of an HDACi and one or more further chemotherapeutic agents. Optionally, the therapeutic regimen may also include additional therapeutic, non-therapeutic or chemotherapeutic agents as described herein. Suitable therapeutic agents include antibodies, such as Avastin, Erbitux and Herceptin, and other therapeutic compounds, such as Tarceva and Tykerb.

In a preferred embodiment, a therapeutic treatment regimen comprises the use of a HDACi, one or more further chemotherapeutic agents, and one or more therapeutic antibodies and/or therapeutic compounds.

In a preferred embodiment, a therapeutic treatment regimen comprises the use of a HDACi, one or more further chemotherapeutic agents, and one or more therapeutic antibodies.

In a preferred embodiment, a therapeutic treatment regimen comprises the use of a HDACi, one or more further chemotherapeutic agents, and one or more therapeutic compounds.

The therapeutic antibody is preferably selected from the group of: Avastin, Erbitux and Herceptin.

The therapeutic agent is preferably selected from the group of Tarceva and Tykerb.

### Modes and Doses of Administration

The HDAC inhibitor can be administered in an oral form, for example, as tablets, capsules (each of which includes sustained release or timed release formulations), pills, powders, granules, elixirs, tinctures, suspensions, syrups, and emulsions, all well known to those of ordinary skill in the pharmaceutical arts. Likewise, the HDAC inhibitor can be administered in intravenous (bolus or infusion), intraperitoneal, subcutaneous, or intramuscular form, well known to those of ordinary skill in the pharmaceutical arts.

The HDAC inhibitor can be administered in the form of a depot injection or implant preparation that can be formulated in such a manner as to permit a sustained release of the active ingredient. The active ingredient can be compressed into pellets or small cylinders and implanted subcutaneously or intramuscularly as depot injections or implants. Implants can employ inert materials such as biodegradable polymers or synthetic silicones, for example, Silastic, silicone rubber or other polymers manufactured by the Dow-Corning Corporation.

The HDAC inhibitor can also be administered in the form of liposome delivery systems, such as small unilamellar vesicles, large unilamellar vesicles and multilamellar vesicles. Liposomes can be formed from a variety of phospholipids, such as cholesterol, stearylamine, or phosphatidylcholines.

The HDAC inhibitor can also be delivered by the use of monoclonal antibodies as individual carriers to which the compound molecules are coupled.

The HDAC inhibitor can also be prepared with soluble polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues.

Furthermore, the HDAC inhibitor can be prepared with biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates, and cross linked or amphipathic block copolymers of hydrogels.

The dosage regimen utilizing the HDAC inhibitor can be selected in accordance with a variety of factors including type, species, age, weight, sex and the type of cancer being treated; the severity (i.e., stage) of the cancer to be treated; the route of administration; the renal and hepatic function of the subject; and the particular compound or salt thereof employed. An ordinarily skilled physician or veterinarian can readily determine and prescribe the effective amount of the drug required to treat, for example, to prevent, inhibit (fully or partially) or arrest the progress of the disease.

Oral dosages of the HDAC inhibitor, when used to treat the desired cancer can range between about 2 mg to about 6000 mg per day, such as from about 20 mg to about 6000 mg per day, such as from about 200 mg to about 6000 mg per day. For example, oral dosages can be about 2, about 20, about 200, about 400, about 800, about 1200, about 1600, about 2000, about 4000, about 5000 or about 6000 mg per day. It is understood that the total amount per day can be administered in a single dose or can be administered in multiple dosing such as twice, three or four times per day.

For example, a subject can receive between about 2 mg/day to about 2000 mg/day, for example, from about 20 to about 2000 mg/day, such as from about 200 to about 2000 mg/day, for example from about 400 mg/day to about 1200 mg/day. A suitably prepared medicament for once a day administration can thus contain between about 2 mg and about 2000 mg, such as from about 20 mg to about 2000 mg, such as from about 200 mg to about 1200 mg, such as from about 400 mg/day to about 1200 mg/day. The HDAC inhibitor can be administered in a single dose or in divided doses of two, three, or four times daily. For administration twice a day, a suitably prepared medicament would therefore contain half of the needed daily dose.

Intravenously or subcutaneously, the subject would receive the HDAC inhibitor (e.g., PXD-101) in quantities sufficient to deliver between about 3-1500 mg/m² per day, for example, about 3, 30, 60, 90, 180, 300, 600, 900, 1000, 1200, or 1500 mg/m² per day. Such quantities can be administered in a number of suitable ways, e.g., large volumes of low concentrations of HDAC inhibitor during one extended period of time or several times a day. The quantities can be administered for one or more consecutive days, intermittent days, or a combination thereof per week (7 day period). Alternatively, low volumes of high concentrations of HDAC inhibitor during a short period of time, e.g., once a day for one or more days either consecutively, intermittently, or a combination thereof per week (7 day period). For example, a dose of 300 mg/m² per day can be administered for 5 consecutive days for a total of 1500 mg/m² per treatment. In another dosing regimen, the number of consecutive days can also be 5, with treatment lasting for 2 or 3 consecutive weeks for a total of 3000 mg/m² and 4500 mg/m² total treatment.

Typically, an intravenous formulation can be prepared which contains a concentration of HDAC inhibitor of from about 1.0 mg/mL to about 10 mg/mL, e.g., 2.0 mg/mL, 3.0 mg/mL, 4.0 mg/mL, 5.0 mg/mL, 6.0 mg/mL, 7.0 mg/mL, 8.0 mg/mL, 9.0 mg/mL, or 10 mg/mL, and administered in amounts to achieve the doses described above. In one example, a sufficient volume of intravenous formulation can be administered to a subject in a day such that the total dose for the day is between about 300 and about 1200 mg/m².

In a preferred embodiment, 1000 mg/m² of PXD-101 is administered intravenously once daily by 30-minute infusion every 24 hours for at least five consecutive days.

In one embodiment, PXD-101 is administered in a total daily dose of up to 1500 mg/m2.

In one embodiment, PXD-101 is administered intravenously in a total daily dose of 1000 mg/m², or 1400 mg/m² or 1500 mg/m², for example, once daily, continuously (every day), or intermittently. In one embodiment, PXD-101 is administered every day on days 1 to 5 every three weeks.

Glucuronic acid, L-lactic acid, acetic acid, citric acid, or any pharmaceutically acceptable acid/conjugate base with reasonable buffering capacity in the pH range acceptable for intravenous administration of the HDAC inhibitor can be used as buffers. Sodium chloride solution wherein the pH has been adjusted to the desired range with either acid or base, for example, hydrochloric acid or sodium hydroxide, can also be employed. Typically, a pH range for the intravenous formulation can be in the range of from about 5 to about 12. A preferred pH range for intravenous formulation wherein the HDAC inhibitor has a hydroxamic acid moiety (e.g., as in PXD-101), can be about 9 to about 12. Consideration should be given to the solubility and chemical compatibility of the HDAC inhibitor in choosing an appropriate excipient.

Subcutaneous formulations, preferably prepared according to procedures well known in the art at a pH in the range between about 5 and about 12, also include suitable buffers and isotonicity agents. They can be formulated to deliver a daily dose of HDAC inhibitor in one or more daily subcutaneous administrations, e.g., one, two or three times each day. The choice of appropriate buffer and pH of a formulation, depending on solubility of the HDAC inhibitor to be administered, is readily made by a person having ordinary skill in the art. Sodium chloride solution wherein the pH has been adjusted to the desired range with either acid or base, for example, hydrochloric acid or sodium hydroxide, can also be employed in the subcutaneous formulation. Typically, a pH range for the subcutaneous formulation can be in the range of from about 5 to about 12. A preferred pH range for subcutaneous formulation wherein the HDAC inhibitor has a hydroxamic acid moiety is about 9 to about 12. Consideration should be given to the solubility and chemical compatibility of the HDAC inhibitor in choosing an appropriate excipient.

The HDAC inhibitor can also be administered in intranasal form via topical use of suitable intranasal vehicles, or via transdermal routes, using those forms of transdermal skin patches well known to those of ordinary skill in that art. To be administered in the form of a transdermal delivery system, the administration will likely be continuous rather than intermittent throughout the dosage regime.

The further chemotherapeutic agent (or agents, if more than one is employed) may be administered using conventional methods and protocols well known to those of skill in the art. For example, a typical dosage rate for 5-fluorouracil (5-FU) is 750-1000 mg/m² in a 24 hour period, administered for 4-5 days every 3 weeks. A typical dose rate for capecitabine is 1000 to 1250 mg/m² orally, when administered twice daily on days 1 to 14 of every 3^{rd} week.

### Pharmaceutical Compositions

The HDAC inhibitor can be administered as an active ingredient in admixture with suitable pharmaceutical diluents, excipients, or carriers (collectively referred to herein as “carrier” materials) suitably selected with respect to the intended form of administration, that is, oral tablets, capsules, elixirs, syrups and the like, and consistent with conventional pharmaceutical practices.

For example, in one embodiment, the pharmaceutical composition comprises the HDAC inhibitor PXD-101 in solution with L-arginine. To prepare this composition, a 10 g quantity of L-arginine was added to a vessel containing approximately 70 mL of Water-For-Injections BP. The mixture was stirred with a magnetic stirrer until the arginine had dissolved. A 5 g quantity of PXD-101 was added, and the mixture stirred at 25° C. until the PXD-101 had dissolved. The solution was diluted to a final volume of 100 mL using Water-For-Injections BP. The resulting solution had a pH of 9.2-9.4 and an osmolality of approximately 430 mOSmol/kg. The solution was filtered through a suitable 0.2 μm sterilizing (e.g., PVDF) membrane. The filtered solution was placed in vials or ampoules, which were sealed by heat, or with a suitable stopper and cap. The solutions were stored at ambient temperature, or, more preferably, under refrigeration (e.g., 2-8° C.) in order to reduced degradation of the drug.

In one embodiment, the HDAC inhibitor (e.g., PXD-101) can be administered orally. Oral administration can be in the form of a tablet or capsule. The HDAC inhibitor can be combined with an oral, non-toxic, pharmaceutically acceptable, inert carrier such as lactose, starch, sucrose, glucose, methyl cellulose, microcrystalline cellulose, sodium croscarmellose, magnesium stearate, dicalcium phosphate, calcium sulfate, mannitol, sorbitol and the like or a combination thereof. For oral administration in liquid form, the HDAC inhibitor can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn-sweeteners, natural and synthetic gums such as acacia, tragacanth or sodium alginate, carboxymethylcellulose, microcrystalline cellulose, sodium croscarmellose, polyethylene glycol, waxes and the like. Lubricants suitable for use in these dosage forms include sodium oleate, sodium stearate, magnesium stearate, sodium benzoate, sodium acetate, sodium chloride, and the like. Disintegrators suitable for use in these dosage forms include starch methyl cellulose, agar, bentonite, xanthan gum and the like.

Suitable pharmaceutically acceptable salts of the HDAC inhibitors described herein, and suitable for use in the method of the invention, are conventional non-toxic salts and can include a salt with a base or an acid addition salt such as a salt with an inorganic base, for example, an alkali metal salt (e.g., lithium salt, sodium salt, potassium salt, etc.), an alkaline earth metal salt (e.g., calcium salt, magnesium salt, etc.), an ammonium salt; a salt with an organic base, for example, an organic amine salt (e.g., triethylamine salt, pyridine salt, picoline salt, ethanolamine salt, triethanolamine salt, dicyclohexylamine salt, N,N′-dibenzylethylenediamine salt, etc.) etc.; an inorganic acid addition salt (e.g., hydrochloride, hydrobromide, sulfate, phosphate, etc.); an organic carboxylic or sulfonic acid addition salt (e.g., formate, acetate, trifluoroacetate, maleate, tartrate, methanesulfonate, benzenesulfonate, p-toluenesulfonate, etc.); a salt with a basic or acidic amino acid (e.g., arginine, aspartic acid, glutamic acid, etc.) and the like.

Various further aspects and embodiments of the present invention will be apparent to those skilled in the art in view of the present disclosure. All documents and database entries mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures and tables.

### EXAMPLES

### Patient Treatment

Thirty-five patients (pts) (Table 1) were treated at different dose-levels of Bel (PXD-101) (mg/m²/30-m in daily infusion)/FU (mg/m²/24 h-infusion): 300/250 (n=4), 600/250 (n=3), 1000/250 (n=6), 1000/500 (n=6), 1000/750 (n=7), 1000/1000 (n=8), 600/1000 (n=1; non-planned FU dose).

Median treatment duration for all patients was 2 cycles (range 1-14). Reasons for discontinuations were progressive disease (25 patients; 71%), adverse events (AEs) (5 patients; 14%; events of pulmonary embolism, vomiting, hypersensitivity, mucosal inflammation, and fatigue), and patient/physician decision (5 patients; 14%).

In total 3 dose limiting toxicities (DLTs) (all in cycle 2) were noted at dose-levels 1000/1000 (2 patients; grade 3 chest pain, grade 3 stomatitis) and 1000/750 (1 pat; grade 3 vomiting), based on which the recommended dose-level was set to 1000/750.

Most frequent AEs (any grade, irrespective of relationship, all patients treated) were: fatigue (80%), nausea (74%), and vomiting (63%) (Table 2). The only grade ¾ treatment related adverse event occurring in more than one pat was fatigue (2 patients; 6% of all patients), and there was only one related serious adverse event (grade 3 fatigue in one pat).

### Cardiac Safety

Cardiac safety was analyzed by extensive ECG monitoring (>3.000 ECG's assessed) in combination with PK assessments of Bel. Based on analyses by a central laboratory, the main findings (mean changes from baseline at the different dose-levels and for Bel alone and in combination with FU) were:
Heart rate, changes of −1 to +11 bpm, without outliers or any dose relationship.
PR duration, changes of −8 to +2 ms, without outliers or any dose relationship.
QRS duration, changes of −3 to +4 ms, without outliers or any dose relationship.
QTcF duration, changes of −4 to +18 ms, without any dose relationship. One outlier pat with a new QTcF value >500 ms was identified at dose-level 1000/750 (max post-infusion QTcF value 522 ms from pre-infusion value of 487 ms).

The lack of dose-relationship and assessment of the slopes of the associated PK-PD model suggests that no effect on cardiac re-polarization was clearly shown. Also, a view of the baseline to each time point analysis demonstrates a lack of time dependent changes in QTcF. Thus, based on central laboratory review and the available information (n=35 patients) the preponderance of the data show that there is no clear signal of any clinically relevant effect on heart rate, AV conduction, cardiac depolarization, morphology or cardiac re-polarization.

### Pharmacokinetics

Bel exhibited linear PK on day 1 across all dose cohorts (n=22). At the recommended dose-level of belinostat (1000 mg/m²/day belinostat; see Table 3), non-significant increases of mean exposure parameters (Cₘₐₓ and AUC), and non-significant decreases of the volume of distribution and clearance, were observed on day 5 compared to day 1.

### Efficacy—Pharmacodynamics

Nine patients (26%) had stable disease, including 6 patients for 4 to 14 cycles (Table 4).

Expression of TS in tumor tissue was down-regulated during Bel monotherapy in 4 of 4 patients having both pre- and post-Bel analyzable samples (Table 5; all patients treated at Bel 1000 mg/m²/day). Three of 4 patients showed up-regulation of p21 (indicating a G1 cell cycle arrest), and minor changes were seen in DPD expression. In addition to patients shown in Table 5, two further patients had biopsies taken from liver lesions (patients 101-11 and 101-12), but these biopsies did not include sufficient material for analyses, either before or after Bel exposure.

Expression of TS, DPD, and p21 was also assessed in surrogate non-tumor tissue, i.e. PBMC (Table 5B). Transforming “raw numbers” of relative gene expression to percentage change from baseline yields an outcome indicating that few patients have a response pattern in surrogate tissue reflecting what might be assumed to be the most favorable pattern for a potentially increased effect of FU due to Bel induced changes of expression levels, i.e. TS down-, DPD down-, and p21 up-regulation. Clinical outcome of patients treated with the combination of Bel and FU seems to indicate that clinical benefit (e.g. long stabilization of disease despite intense pre-treatment) might be linked to a favorable Bel impact on expression of TS, DPD, and p21 (Table 6).

Among the 20 patients where an attempt was made to analyze TS, DPD, and p21 in PBMC, 2 patients seemed to have a clearly favorable clinical outcome (patients 101-3 and 102-14 with treatment durations on Bel/FU of 8 and 14 cycles, respectively; see Tables 4 and 6), i.e. a 10% favorable outcome rate without selection.

If selection of patients for treatment with Bel/FU would have been done after an initial-dose of Bel and analyses of TS, DPD, and p21 expression in PBMC 6-hours post Bel dosing, the favorable outcome rate could have been increased to between 33% and 67% by selection of patients based on a “2 out of 3” positive expression pattern (positive assumed to be TS down-, DPD down-, and p21 up-regulation in comparison to a pre-treatment value).

Selection based on one single sample taken 6-hours post Bel dosing (Table A): 6 (30%; patients 101-3/5/6/7, 102-6/14) of the 20 patients have “2 out of 3” markers showing a favorable expression change after Bel dosing, and 2 (33%) of the 6 patients have clearly favorable clinical outcomes. Note, also patients 101-6 and 101-7 with a “2 out of 3” pattern, but not counted as having “clearly favorable clinical outcome” have longer than median treatment durations on Bel/FU (i.e. 4 cycles; see Table 4).

Selection based on mean of two samples taken 6-hours post Bel dosing (Table B): 3 (15%; patients 101-3, 102-6/14) of the 20 patients have “2 out of 3” markers showing a favorable expression change after Bel dosing, and 2 (67%) of the 3 patients have clearly favorable clinical outcomes.

Extending the number of sampling times does not seem to increase predictability significantly, e.g. data presented in Table 6 showing maximum percentage change during Bel treatment in cycle 1 based on four sampling time points (day 1 and 4, each day 6-hours and 24-hours post Bel dosing) versus baseline indicates that 5 (25%) of the 20 patients have “2 out of 3” markers showing a favorable expression change after Bel dosing, and 2 (40%) of the 5 patients have favorable clinical outcomes.

Evaluating one single PBMC sample 6-hours post Bel dosing vs a pre-Bel sample in the 20 patients:

Six (30%; patients 101-3/5/6/7, 102-6/14) of the 20 patients have “2 of 3” markers showing a favorable expression change after Bel dosing, and 2 (33%) of the 6 patients have a clearly favorable clinical outcome.

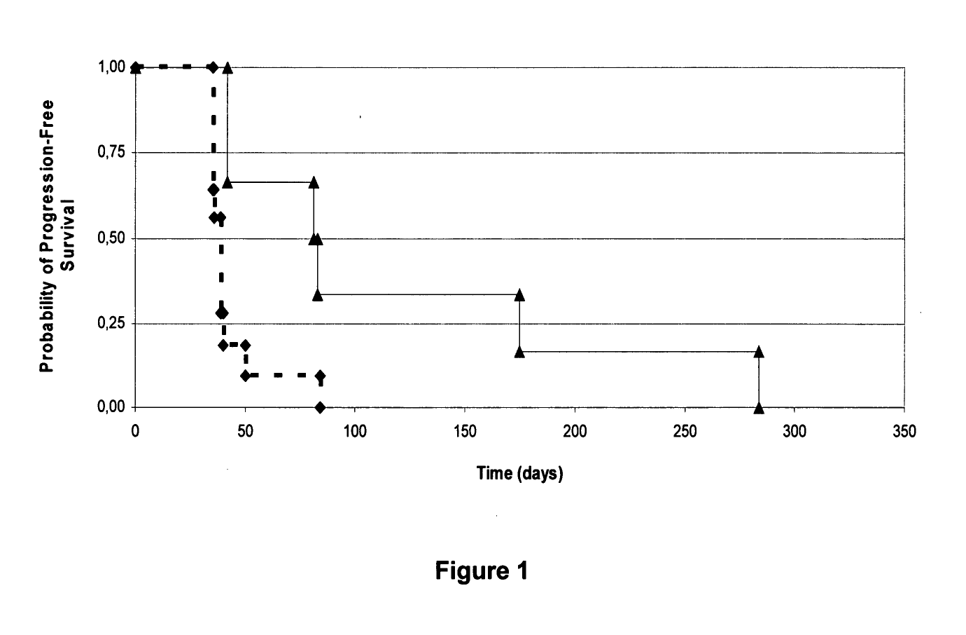

Comparing the 6 patients with a “2 of 3” marker pattern versus the 14 patients without such a pattern shows that progression-free survival (PFS) on Bel/FU is significantly (p<0.04) longer in patients with the “2 of 3” marker pattern (FIG. 1).

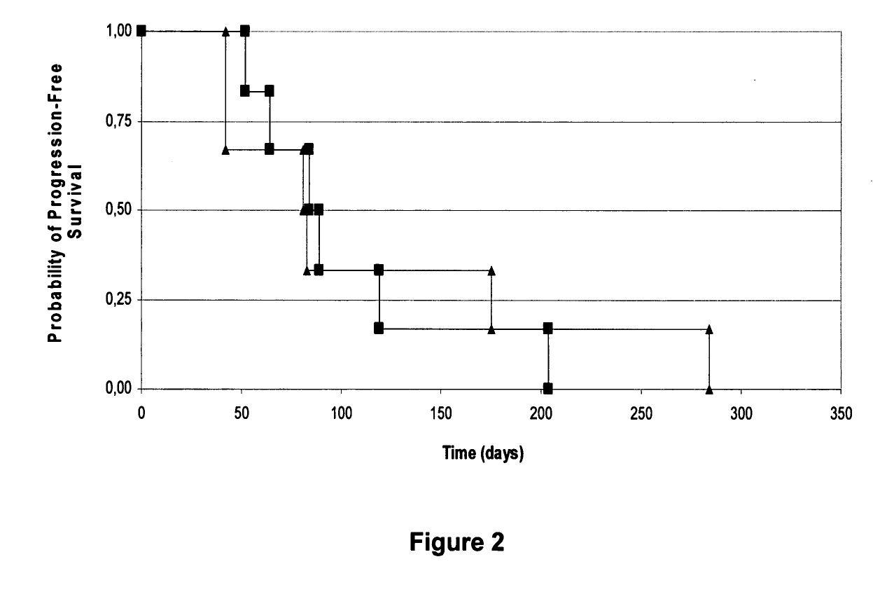

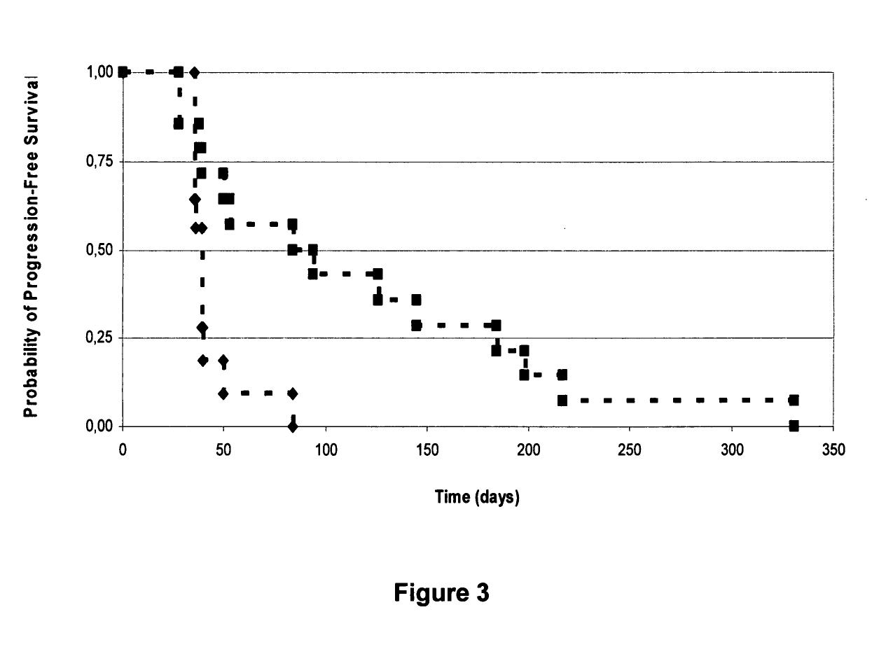

Although patients with and without a “2 of 3” marker pattern have similarly long PFS on their most recently received pre-treatments before Bel/FU, there is a striking difference comparing each groups PFS on Bel/FU to PFS on their respective most recent pre-treatments (FIGS. 2 and 3). Patients with a “2 of 3” marker pattern after one single dose of Bel have a PFS on Bel/FU similar to what could be achieved with their most recent previous treatment.

### Methods

Phase I dose-escalation, multi-center study with the objectives to assess:
maximum tolerated dose and dose limiting toxicities (DLT) of Bel in combination with FU
impact of Bel on expression of TS, dihydropyrimidine dehydrogenase (DPD), and p21, in tumor and non-tumor surrogate tissue (peripheral blood mononuclear cells; PBMC)
preliminary anti-tumor activity of Bel in combination with FU
impact of Bel on ECG parameters
pharmacokinetics (PK) of Bel when given in combination with FU

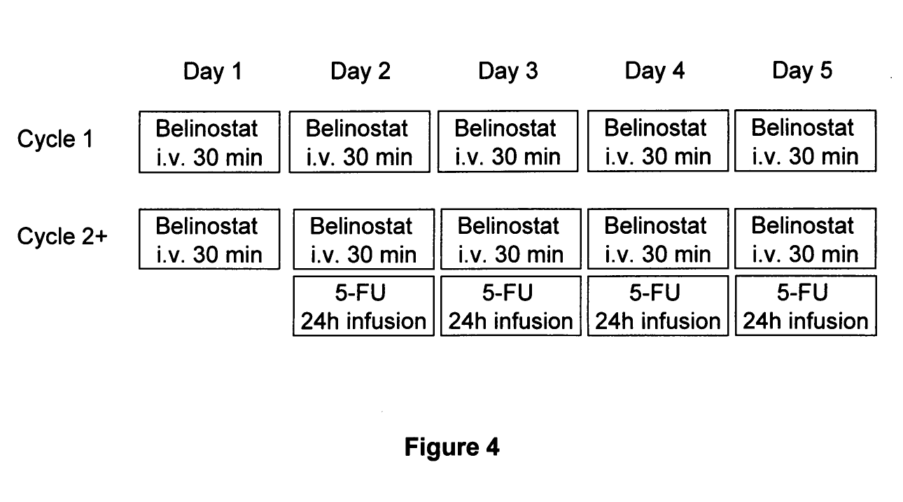

Treatment cycles of 21 days included Bel alone in cycle 1, and Bel in combination with FU in all subsequent cycles (FIG. 4). Treatment was to continue until significant treatment-related toxicity or progressive disease.

Patients with advanced solid tumors with progression after standard chemotherapy were included. Other main criteria for inclusion included measurable disease, Karnofsky performance >70%, acceptable organ functions, no significant cardiovascular disease, baseline QTc interval <500 ms, and no known active HIV, hepatitis B, hepatitis C, or infection requiring IV treatment.

DLTs, i.e. grade ¾ non-hematologic toxicities and grade 4 neutropenia or thrombocytopenia, were determined in cycles 1 and 2. Safety was assessed by NCI CTC (v. 3) and efficacy by RECIST criteria. Expression of TS, DPD, and p21 mRNA was measured by RTQ-PCR in PBMC (20 patients at baseline, and pre-dosing, 6 and 24-hours post-dosing, on days 1 and 4 in cycle 1) and in tumor biopsies (8 patients at baseline and once on days 4 or 5 in cycle 1). Results were expressed as gene expression relative to actin (i.e. TS/Actin Relative Gene Expression, DPD/Actin Relative Gene Expression, and p21/Actin Relative Gene Expression).

### CONCLUSION

The recommended schedule of belinostat in combination with 5-FU (BelFU) was determined to be belinostat 1000 mg/m²/day administered by 30-min infusions once daily days 1-5 and 5-FU 750 mg/m²/24 h by a 96-hours continuous infusion starting day 2.

The combination was shown to be safe, and no un-expected adverse events were seen. Based on central laboratory review of extensive ECG monitoring it was shown that there is no clear signal of any clinically relevant effect on heart rate, AV conduction, cardiac depolarization, morphology or cardiac re-polarization during belinostat or BelFU treatment.

Despite the extensive pre-treatment (median of 3 prior regimens; majority of patients treated with 2 or more FU-based regimens) 26% of patients on BelFU achieved a stabilization of disease, including 6 patients with time to progression of 12 to 41 weeks.

The pre-clinically shown down-regulation of thymidylate synthase (TS; main target of FU) by belinostat was confirmed in the clinic. Four of 4 assessable patients showed down-regulation of TS in tumor tissue during belinostat monotherapy, and 3 of the 4 patients also had an up-regulation of p21 (indicating a G1 cell cycle arrest).

Assessment of belinostat impact on TS, dihydropyrimidine dehydrogenase (DPD), and p21 expression in surrogate non-tumor tissue, i.e. PBMC, indicated a potential for an outcome-linked favorable expression pattern consisting of TS down-, DPD down-, and p21 up-regulation. Clinical outcome of patients treated with BelFU who after a single dose of belinostat showed an expression pattern including a favorable change of “2 of 3” markers in PBMC, had significantly superior PFS in comparison to patients not having this expression pattern.

The BelFU combination should be further evaluated, preferably in patients with less advanced pre-treatment, including further assessments of the potential for patient selection based on a favourable expression pattern change for TS, DPD, and p21, after exposure of patient PBMC's to belinostat (either as a clinical “test-dose” or potentially in vitro).

TABLE 1
Baseline characteristics

| Characteristic | N = 35 |
|---|---|
| Age (years) | |
| Median (range) | 68 (36-81) |
| Gender (%) | |
| Male/Female | 54%/46% |
| Karnofsky PS (%) | |
| 100 | 43% |
| 90 | 29% |
| ≦80 | 29% |
| Diagnosis (%) | |
| Colorectal | 40% |
| Pancreatic | 14% |
| Esophageal/Gastric | 11% |
| Head & neck | 9% |
| Prostate | 6% |
| Other cancer | 20% |
| Number of Prior Chemo Regimens | |
| Median (range) | 3 (1-10) |
| Number of Prior Fluoropyrimidine | |
| Containing Regimens (%) | |
| 0 | 23% |
| 1 | 26% |
| ≧2 | 52% |
| (2-3-4-5 FU regimens) | (26%-6%-14%-6%) |

TABLE 2
Adverse events (irrespective of relationship) reported in >20%
of all pts or pts treated at recommended dose-level (Bel/FU: 1000/750)

| | All Pts | | | | | Pts at dose-level 1000/750 | | | | |
|---|---|---|---|---|---|---|---|---|---|---|
| | (n = 35) | | | | | (n = 7) | | | | |
| | All grade | Gr 1 | Gr 2 | Gr 3 | Gr 4 | All grade | Gr 1 | Gr 2 | Gr 3 | Gr 4 |
| | No (%) | No | No | No | No | No (%) | No | No | No | No |
| Fatigue | 28 (80) | 8 | 14 | 5 | 1 | 3 (43) | 0 | 3 | 0 | 0 |
| Nausea | 26 (74) | 15 | 11 | 0 | 0 | 6 (86) | 2 | 4 | 0 | 0 |
| Vomiting | 22 (63) | 12 | 9 | 1 | 0 | 4 (57) | 2 | 1 | 1 | 0 |
| Anorexia | 15 (43) | 6 | 9 | 0 | 0 | 2 (29) | 2 | 0 | 0 | 0 |
| Constipation | 15 (43) | 11 | 4 | 0 | 0 | 2 (29) | 2 | 0 | 0 | 0 |
| Diarrhea | 12 (34) | 4 | 8 | 0 | 0 | 3 (43) | 0 | 3 | 0 | 0 |
| Dysgeusia | 11 (31) | 8 | 3 | 0 | 0 | 1 (14) | 0 | 1 | 0 | 0 |
| Anemia | 10 (29) | 3 | 6 | 1 | 0 | 0 | 0 | 0 | 0 | 0 |
| Dyspnoea | 10 (29) | 5 | 5 | 0 | 0 | 1 (14) | 0 | 1 | 0 | 0 |
| Dehydration | 9 (26) | 0 | 7 | 2 | 0 | 0 | 0 | 0 | 0 | 0 |
| Pyrexia | 8 (23) | 5 | 3 | 0 | 0 | 1 (14) | 1 | 0 | 0 | 0 |
| Abd pain | 7 (20) | 1 | 5 | 1 | 0 | 2 (29) | 0 | 1 | 1 | 0 |
| Dizziness | 7 (20) | 4 | 3 | 0 | 0 | 2 (29) | 1 | 1 | 0 | 0 |
| Headache | 7 (20) | 7 | 0 | 0 | 0 | 1 (14) | 1 | 0 | 0 | 0 |
| Chills | 6 (17) | 6 | 0 | 0 | 0 | 2 (29) | 2 | 0 | 0 | 0 |
| Flushing | 4 (11) | 3 | 1 | 0 | 0 | 2 (29) | 2 | 0 | 0 | 0 |
| Dysphonia | 2 (6) | 2 | 0 | 0 | 0 | 2 (29) | 2 | 0 | 0 | 0 |

TABLE 3
Group mean (±SD) PK parameters from pts receiving belinostat
1000 mg/m²/day alone on day 1 and in combination with FU on day 5

| | Cycle 2 Day 1 | Cycle 2 Day 5 |
|---|---|---|
| | n = 14 | n = 13 |
| T_{1/2} (hr) | 1.0 (0.3) | 1.2 (0.3) |
| Cₘₐₓ (ng/ml) | 42,657 (11,281) | 49,500 (16,626) |
| AUCₐₗₗ (hr*ng/ml) | 28,93 (9,238) | 33,199 (10,633) |
| AUC_{0-∞} (hr*ng/ml) | 29,005 (9,295) | 33,295 (10,676) |
| CL (ml/hr/m²) | 37,580 (10,893) | 30,141 (13,824) |
| Vₛₛ (ml/m²) | 16,528 (4,918) | 13,538 (7,253) |
| V_{z} (ml/m²) | 58,362 (26,136) | 53,316 (29,753) |

TABLE 4
Pts with stabilization of disease for ≧4 treatment cycles of belinostat in combination with 5-FU

| | Pat | Pat | Pat | Pat | Pat | Pat |
|---|---|---|---|---|---|---|
| | 101-3 | 101-6 | 103-3 | 101-7 | 102-14 | 102-15 |
| Gender/Age | F/63 | M/60 | F/69 | F/62 | F/73 | M/72 |
| Primary Site (all adenoca) | Colon | Esophageal | Pancreatic | Breast | Colon | Pancreatic |
| Disease localization | lung | pleura, | liver, | liver, | liver, lung, | lung, |
| | | lymph nodes | lymph nodes | lymph nodes, | kidney, abd | lymph nodes |
| | | | | spleen, bone | wall, psoas | |
| | | | | | muscle | |
| Prior lines of therapy | 3 | 3 | 3 | 10 | 4 | 3 |
| Most recent prior FU-based | FOLFOX-4 | cisplatin/ | capecitabine | capecitabine | capecitabine/ | 5-FU (CIV) |
| line of therapy (trt dura- | (8.7 weeks) | 5-FU | (31.3 weeks) | (23.0 weeks) | oxaliplatin | (4.0 weeks) |
| tion; TTP not available) | | (5.4 weeks) | | | (17.6 weeks) | |
| Most recent prior line of | irinotecan/ | paclitaxel | erlotinib/ | Abraxane/ | irinotecan/ | investiga- |
| therapy (TTP) | cetuximab | (12.0 weeks) | gemcitabine | bevacizumab | cetuximab | tional drug |
| | (7.4 weeks) | | (51.1 weeks) | (12.7 weeks) | (9.1 weeks) | (5.4 weeks) |
| Study treatment | | | | | | |
| Dose-level | 600/1000 | 1000/250 | 1000/500 | 1000/1000 | 1000/1000 | 1000/1000 |
| Best overall response | SD (−9%) | SD (0%) | SD (−5%) | SD (+2%) | SD (−9%) | SD (+1%) |
| (max % target lesion change before PD) | | | | | | |
| No of treatment cycles | 8 | 4 | 4 | 4 | 14 | 4 |
| Reason trt termination | PD | PD | Pat req. | PD | PD | PD |
| Time to progression (TTP) | 25.0 weeks | 11.9 weeks | 12.1 weeks | 11.6 weeks | 40.6 weeks | 12.0 weeks |
| TTP on BelFU vs on most | [symbol] / [symbol] | [symbol] /≈ | [symbol] / [symbol] | [symbol] /≈ | [symbol] / [symbol] | [symbol] / [symbol] |
| recent prior FU-based/ | | | | | | |
| most recent prior therapy | | | | | | |

TABLE 5
Thymidylate synthase (TS), dihydropyrimidine dehydrogenase (DPD), and p21
relative gene expression in patient tumor samples pre- and post-belinostat
treatment [post = cycle 1 day 4 or 5; NST = not sufficient tumor sample]

| | TS | | | DPD | | | p21 | | |
|---|---|---|---|---|---|---|---|---|---|
| | | | Change | | | Change | | | Change |
| Patient No | Pre- | Post- | vs | Pre- | Post- | vs | Pre- | Post- | vs |
| Biopsy location | Bel | Bel | baseline | Bel | Bel | baseline | Bel | Bel | baseline |
| 101-13 | 5.15 | 2.54 | [symbol] | 0.45 | 0.46 | ≈ | 10.42 | 18.49 | [symbol] |
| Lymph node | | | | | | | | | |
| 102-16 | 4.06 | 2.94 | [symbol] | 0.58 | 0.23 | [symbol] | 9.20 | 17.79 | [symbol] |
| Liver | | | | | | | | | |
| 102-17 | NST | 3.18 | — | NST | 0.32 | — | NST | 67.39 | — |
| Liver | | | | | | | | | |
| 103-4 | NST | 4.35 | — | NST | 0.80 | — | NST | 10.16 | — |
| Liver | | | | | | | | | |
| 103-5 | 4.37 | 1.05 | [symbol] | 0.14 | 0.73 | [symbol] | 12.76 | 31.76 | [symbol] |
| Abdominal wall | | | | | | | | | |
| 103-2 | 8.99 | 7.01 | [symbol] | 0.68 | 0.95 | [symbol] | 15.94 | 16.95 | ≈ |
| Liver | | | | | | | | | |

TABLE 5B
Thymidylate synthase (TS), dihydropyrimidine dehydrogenase (DPD), and p21 relative gene
expression in patient peripheral blood mononuclear cells (PBMC) pre- and post-belinostat
treatment [each value is mean of 2 samples; post-belinostat samples drawn day 1 and 4]

| Patient No | TS | | | DPD | | | p21 | | |
|---|---|---|---|---|---|---|---|---|---|
| Bel dose | | 6-hrs | 24-hrs | | 6-hrs | 24-hrs | | 6-hrs | 24-hrs |
| (mg/m²/day) | Baseline | post-Bel | post-Bel | Baseline | post-Bel | post-Bel | Baseline | post-Bel | post-Bel |
| 101-1/300 | 0.1 | 0.7 | 0.2 | 5.8 | 13.4 | 3.6 | 2.9 | 4.1 | 3.3 |
| 101-2/600 | 0.3 | 0.8 | 0.6 | 8.5 | 6.6 | 7.3 | 4.1 | 3.4 | 5.0 |
| 101-3/600 | 0.6 | 0.2 | 0.4 | 9.4 | 3.9 | 9.0 | 2.8 | 3.3 | 4.3 |
| 102-4/600 | 0.2 | 0.4 | NA | 10.8 | 6.8 | 4.7 | 11.2 | 5.0 | 1.9 |
| 102-5/600 | NA | NA | NA | 4.8 | 4.2 | NA | NA | 1.7 | NA |
| 101-4/1000 | 0.2 | 0.6 | 0.3 | 4.8 | 22.8 | 5.6 | 4.6 | 6.1 | 5.0 |
| 102-6/1000 | 0.4 | 0.2 | 0.3 | 4.6 | 2.1 | 8.8 | 0.9 | 3.3 | 9.6 |
| 102-7/1000 | NA | 0.5 | NA | 1.2 | 1.3 | 3.0 | 6.7 | 3.0 | 5.2 |
| 102-8/1000 | 0.8 | 1.2 | 0.5 | 13.0 | 29.1 | 21.5 | 1.3 | 3.2 | 5.6 |
| 101-5/1000 | 0.5 | 1.9 | 0.4 | 6.8 | 5.3 | 5.6 | 2.0 | 1.6 | 2.5 |
| 101-6/1000 | 0.1 | 0.2 | 0.2 | 3.4 | 12.5 | 7.1 | 3.1 | 4.5 | 5.5 |
| 102-9/1000 | NA | NA | NA | NA | NA | NA | NA | NA | NA |
| 102-10/1000 | NA | 1.2 | NA | 1.9 | 7.3 | NA | 1.6 | 1.9 | NA |
| 102-11/1000 | NA | 0.1 | NA | 2.0 | 3.2 | NA | 1.3 | 1.0 | NA |
| 101-7/1000 | 0.7 | 1.0 | 1.0 | 12.2 | 14.0 | 11.2 | 7.3 | 4.7 | 4.8 |
| 101-8/1000 | 0.3 | 0.8 | 0.5 | 8.9 | 15.3 | 12.6 | 5.5 | 6.8 | 1.1 |
| 102-12/1000 | NA | NA | NA | NA | 2.3 | 1.2 | NA | 3.8 | 2.0 |
| 102-13/1000 | NA | 0.2 | 1.0 | NA | 15.3 | 1.8 | NA | 0.6 | NA |
| 102-14/1000 | 1.2 | NA | 0.4 | 0.9 | 0.7 | 0.7 | 2.4 | 4.2 | 3.5 |
| 102-15/1000 | 0.4 | NA | 1.1 | 2.3 | NA | 3.7 | 2.2 | NA | 1.2 |
| Median | 0.4 | 0.6 | 0.4 | 4.8 | 6.7 | 5.6 | 2.9 | 3.4 | 4.3 |
| Min | 0.1 | 0.1 | 0.2 | 0.9 | 0.7 | 0.7 | 0.9 | 0.6 | 1.1 |
| Max | 1.2 | 1.9 | 1.1 | 13.0 | 29.1 | 21.5 | 11.2 | 6.8 | 9.6 |

TABLE 6
Maximum percentage change during belinostat treatment (cycle
1) versus baseline of thymidylate synthase (TS), dihydropyrimidine
dehydrogenase (DPD), and p21 relative gene expression in
patient peripheral blood mononuclear cells

| | Study Trt | | | |
|---|---|---|---|---|
| | Outcome | | | |
| Patient No | No of trt | | | |
| Belinostat Dose | cycles/best | | | |
| (mg/m²/day) | response | TS | DPD | P21 |
| 101-1/300 | 2/PD | 486% | 136% | 48% * |
| 101-2/600 | 2/PD | 190% | −74% * | 43% * |
| 101-3/600 | 8/SD | −87% * | −87% * | 88% * |
| 102-4/600 | 2/PD | 144% | −73% * | −86% |
| 102-5/600 | 2/PD | NA | −13% * | NA |
| 101-4/1000 | 2/PD | 238% | 547% | 68% * |
| 102-6/1000 | 2/PD | −51% * | 91% | 972% * |
| 102-7/1000 | 2/PD | NA | 220% | −72% |
| 102-8/1000 | 2/PD | 63% | 169% | 715% * |
| 101-5/1000 | 2/PD | 594% | −83% * | 62% * |
| 101-6/1000 | 4/SD | 250% | 563% | 156% * |
| 102-9/1000 | 2/PD | NA | NA | NA |
| 102-10/1000 | 2/PD | NA | 373% | 43% * |
| 102-11/1000 | 2/PD | NA | 98% | −71% |
| 101-7/1000 | 4/SD | 116% | −60% * | −83% |
| 101-8/1000 | 2/PD | 274% | 160% | −86% |
| 102-12/1000 | 2/PD | NA | NA | NA |
| 102-13/1000 | 1/NE | NA | NA | NA |
| 102-14/1000 | 14/SD | −67% * | −20% * | 74% * |
| 102-15/1000 | 4/SD | 214% | 62% | −47% |

[* indicates assumed most favourable response for a potentially increased effect of FU due to belinostat impact on expression levels, i.e. TS down-, DPD down-, and p21 up-regulation, respectively]

TABLE A

| Site | Pat | PXD | FU | Cycles sta [symbol] | Response | Reason of [symbol] | D1-6: TS | DPD | p21 |
|---|---|---|---|---|---|---|---|---|---|
| 101 | 1 | 300 | 250 | 2 | PD | vol. withdra [symbol] | 486% | 136% | 35% |
| 101 | 2 | 600 | 250 | 2 | PD | PD | 62% | 30% | −9% |
| 101 | 3 | 600 | 1000 | 8 | SD | PD | −87% | −87% | 31% |
| 102 | 4 | 600 | 250 | 2 | PD | PD | 144% | −56% | −48% |
| 102 | 5 | 600 | 250 | 2 | PD | PD | NA | −13% | NA |
| 101 | 4 | 1000 | 250 | 2 | PD | PD | 238% | 547% | 68% |
| 102 | 6 | 1000 | 250 | 2 | PD | PD | −51% | −55% | 272% |
| 102 | 7 | 1000 | 250 | 2 | PD | PD | NA | −57% | −54% |
| 102 | 8 | 1000 | 250 | 2 | PD | PD | 63% | 78% | 115% |
| 101 | 5 | 1000 | 250 | 2 | PD | PD | −21% | −62% | −25% |
| 101 | 6 | 1000 | 250 | 4 | SD | PD | 88% | −28% | 16% |
| 102 | 10 | 1000 | 500 | 2 | PD | PD | NA | 373% | 43% |
| 102 | 11 | 1000 | 500 | 2 | PD | PD | NA | 14% | 21% |
| 101 | 7 | 1000 | 1000 | 4 | SD | PD | −14% | 6% | 13% |
| 101 | 8 | 1000 | 1000 | 2 | PD | PD | 274% | 160% | −11% |
| 102 | 14 | 1000 | 1000 | 14 | SD | PD | NA | −20% | 74% |
| 102 | 15 | | 1000 | 4 | SD | PD | NA | NA | NA |

Table above is “Change from baseline at 6 h day 1”
“Wished” pattern is “down TS-down DPD-up p21”

| | | |
|---|---|---|
| 2 of 3 correct | pts | 6 |
| Clinical benefit | pts | 2 |
| | Rate | 33% |
| 3 of 3 correct | pts | 2 |
| Clinical benefit | pts | 1 |
| | Rate 1 | 50% |

Without testing: 20 pts (incl 3 tested and all NA outcome), 2 with clinical benefit => Rate 10% [symbol]
indicates data missing or illegible when filed

TABLE B

| Site | Pat | PXD | FU | Cycles sta [symbol] | Response | Reason of [symbol] | 6mean-TS | 6mean-DPD | 6mean-p21 |
|---|---|---|---|---|---|---|---|---|---|
| 101 | 1 | 300 | 250 | 2 | PD | vol. withdra [symbol] | 410% | 131% | 41% |
| 101 | 2 | 600 | 250 | 2 | PD | PD | 126% | −22% | −16% |
| 101 | 3 | 600 | 1000 | 8 | SD | PD | −65% | −59% | 15% |
| 102 | 4 | 600 | 250 | 2 | PD | PD | 144% | −37% | −55% |
| 102 | 5 | 600 | 250 | 2 | PD | PD | NA | −13% | NA |
| 101 | 4 | 1000 | 250 | 2 | PD | PD | 182% | 372% | 32% |
| 102 | 6 | 1000 | 250 | 2 | PD | PD | −51% | −55% | 272% |
| 102 | 7 | 1000 | 250 | 2 | PD | PD | NA | 8% | −55% |
| 102 | 8 | 1000 | 250 | 2 | PD | PD | 54% | 124% | 155% |
| 101 | 5 | 1000 | 250 | 2 | PD | PD | 286% | −22% | −17% |
| 101 | 6 | 1000 | 250 | 4 | SD | PD | 169% | 267% | 44% |
| 102 | 10 | 1000 | 500 | 2 | PD | PD | NA | 278% | 21% |
| 102 | 11 | 1000 | 500 | 2 | PD | PD | NA | 56% | −25% |
| 101 | 7 | 1000 | 1000 | 4 | SD | PD | 51% | 15% | −35% |
| 101 | 8 | 1000 | 1000 | 2 | PD | PD | 142% | 73% | 23% |
| 102 | 14 | 1000 | 1000 | 14 | SD | PD | NA | −20% | 74% |
| 102 | 15 | 1000 | 1000 | 4 | SD | PD | NA | NA | NA |

Table above is “Change from baseline at 6 h based on mean of d1 + d4”
“Wished” pattern is “down TS-down DPD-up p21”

| | | |
|---|---|---|
| 2 of 3 correct | pts | 3 |
| Clinical benefit | pts | 2 |
| | Rate | 67% |
| 3 of 3 correct | pts | 2 |
| Clinical benefit | pts | 1 |
| | Rate 1 | 50% |

Without testing: 20 pts (incl 3 tested and all NA outcome), 2 with clinical benefit => Rate 10% [symbol]
indicates data missing or illegible when filed

1. A method of assessing the susceptibility of a subject to cancer treatment with a therapeutic regimen comprising the use of a histone deacetylase inhibitor (HDACi) and one or more further chemotherapeutic agents, the method comprising:
determining the level of expression of TS, DPD and p21 after administration of an initial dose of HDACi;
wherein a subject susceptible to treatment with the therapeutic regimen comprising the use of a histone deacetylase inhibitor (HDACi) and one or more further chemotherapeutic agents shows at least two of: a decrease in expression of TS, a decrease in expression of DPD, and an increase in expression of p21, after administration of the initial dose of the HDACi.

2. A method according to claim 1, wherein the method further comprises determining the level of expression of TS, DPD and p21 in a sample isolated from a subject prior to administration of an HDACi.

3. A method according to claim 1, wherein the initial dose of the HDACi is administered to a sample isolated from the subject.

4. A method according to claim 1, wherein the method further comprises treating the subject with the histone deacetylase inhibitor (HDACi) and the one or more further chemotherapeutic agents.

5. A method according to claim 1, wherein the level of expression of TS, DPD and p21 is determined using quantitative PCR.

6. A method according to claim 1, wherein the expression of TS, DPD and p21 is determined in a sample obtained from said subject.

7. A method according to claim 1, wherein the expression of TS, DPD and p21 is measured in two samples obtained from said subject.

8. A method according to claim 1, wherein the sample is taken six hours after administration of the initial dose of HDACi to the patient

9. A method according to claim 1, wherein the sample is a peripheral blood mononuclear cell (PBMC) sample, a mucosal sample or a tumour sample.

10. A method according to claim 1, wherein the further chemotherapeutic agent is selected from the group of: fluoropyrimidine compounds, anti-folate compounds, thymidylate synthase (TS) inhibitors, anti-metabolite compounds, and pharmaceutically acceptable salts and solvates thereof.

11. A method according to claim 1, wherein the further chemotherapeutic agent is selected from the group of: 5-fluorouracil (FU), capecitabine, pemetrexed (MTA), pralatrexate (PDX), thymitaq (AG337), plevitrexed (ZD9331), BGC945, raltitrexed, GW1843, methotrexate (MTX), edatrexate (EDX), aminopterin (AMT), PT523, and neutrexin (trimetrexate), UFT and S-1 and pharmaceutically acceptable salts and solvates thereof.

12. A method according to claim 1, wherein the further chemotherapeutic agent is selected from the group of: 5-fluorouracil, pralatrexate, capecitabine and pemetrexed.

13. A method according to claim 1, wherein the cancer is selected from the group of: colorectal cancer, pancreatic cancer, esophagal cancer, gastric cancer, head and neck cancer, prostate cancer, non small cell lung cancer, non-Hodgkin lymphoma and breast cancer.

14. A method according to claim 1, wherein the HDACi is a hydroxamic acid based HDAC inhibitor.

15. A method according to claim 1, wherein the HDACi is selected from PXD-101 (belinostat), vorinostat, panobinostat (hydroxamate), romidepsin (depsipeptide), SNDX-275, MGCD-0103, PCI24781, CHR-3996, ITF2357, SB939, JNJ26481585, JNJ16241199, valproic acid, and pharmaceutically acceptable salts and solvates thereof.

16. A method according to claim 1, wherein the subject shows a decrease in expression of TS, a decrease in expression of DPD, and an increase in expression of p21.

17. A method of assessing the susceptibility of a subject to cancer treatment with a therapeutic regimen comprising the use of a histone deacetylase inhibitor (HDACi) and one or more further chemotherapeutic agents, the method comprising:
(i) administering an initial dose of an HDACi to the subject or to a sample isolated from the subject;
(ii) determining the level of expression of TS, DPD and p21; and
(iii) comparing the level of expression of TS, DPD and p21 before and after administration of the initial dose of the HDACi,
wherein a subject susceptible to treatment with the therapeutic regimen comprising the use of a histone deacetylase inhibitor (HDACi) and one or more further chemotherapeutic agents, shows at least two of a decrease in expression of TS, a decrease in expression of DPD, and an increase in expression of p21, after administration of the initial dose of the HDACi.

18. A method according to claim 17, wherein the method further comprises determining the level of expression of TS, DPD and p21 in a sample isolated from a subject prior to administration of an HDACi.

19. A method according to claim 17, wherein the initial dose of the HDACi is administered to a sample isolated from the subject.

20. A method according to claim 17, wherein the method further comprises treating the subject with the histone deacetylase inhibitor (HDACi) and the one or more further chemotherapeutic agents.

21. A method according to claim 17, wherein the level of expression of TS, DPD and p21 is determined using quantitative PCR.

22. A method according to claim 17, wherein the expression of TS, DPD and p21 is determined in a sample obtained from said subject.

23. A method according to claim 17, wherein the expression of TS, DPD and p21 is measured in two samples obtained from said subject.

24. A method according to claim 17, wherein the sample is taken six hours after administration of the initial dose of HDACi to the patient

25. A method according to claim 17, wherein the sample is a peripheral blood mononuclear cell (PBMC) sample, a mucosal sample or a tumour sample.

26. A method according to claim 17, wherein the further chemotherapeutic agent is selected from the group of: fluoropyrimidine compounds, anti-folate compounds, thymidylate synthase (TS) inhibitors, anti-metabolite compounds, and pharmaceutically acceptable salts and solvates thereof.

27. A method according to claim 17, wherein the further chemotherapeutic agent is selected from the group of: 5-fluorouracil (FU), capecitabine, pemetrexed (MTA), pralatrexate (PDX), thymitaq (AG337), plevitrexed (ZD9331), BGC945, raltitrexed, GW1843, methotrexate (MTX), edatrexate (EDX), aminopterin (AMT), PT523, and neutrexin (trimetrexate), UFT and S-1 and pharmaceutically acceptable salts and solvates thereof.

28. A method according to claim 17, wherein the further chemotherapeutic agent is selected from the group of: 5-fluorouracil, pralatrexate, capecitabine and pemetrexed.

29. A method according to claim 17, wherein the cancer is selected from the group of colorectal cancer, pancreatic cancer, esophagal cancer, gastric cancer, head and neck cancer, prostate cancer, non small cell lung cancer, non-Hodgkin lymphoma and breast cancer.

30. A method according to claim 17 wherein the HDACi is a hydroxamic acid based HDAC inhibitor.

31. A method according to claim 17, wherein the HDACi is selected from PXD-101 (belinostat), vorinostat, panobinostat (hydroxamate), romidepsin (depsipeptide), SNDX-275, MGCD-0103, PCI24781, CHR-3996, ITF2357, SB939, JNJ26481585, JNJ16241199, valproic acid, and pharmaceutically acceptable salts and solvates thereof.

32. A method according to claim 17, wherein the subject shows a decrease in expression of TS, a decrease in expression of DPD, and an increase in expression of p21.

33. A method of treating cancer in a subject, the method comprising:
(i) administering an initial dose of a histone deacetylase inhibitor (HDACi) to the subject or to a sample isolated from the subejct;
(ii) determining the level of expression of TS, DPD and p21;
(iii) comparing the level of expression of TS, DPD and p21 before and after administration of the initial dose of the HDACi; and
(iv) administering a therapeutically effective amount of a therapeutic regimen comprising an HDACi and one or more further chemotherapeutic agents to the subject,
provided that the subject showed at least two of: a decrease in expression of TS, a decrease in expression of DPD, and an increase in expression of p21 after administration of the initial dose of the HDACi.

34. A method according to claim 33, wherein the level of expression of TS, DPD and p21 is determined using quantitative PCR.

35. A method according to claim 33, wherein the expression of TS, DPD and p21 is determined in a sample obtained from said subject.

36. A method according to claim 33, wherein the expression of TS, DPD and p21 is measured in two samples obtained from said subject.

37. A method according to claim 33, wherein the sample is taken six hours after administration of the initial dose of HDACi to the patient

38. A method according to claim 33, wherein the sample is a peripheral blood mononuclear cell (PBMC) sample, a mucosal sample or a tumour sample.

39. A method according to claim 33, wherein the further chemotherapeutic agent is selected from the group of: fluoropyrimidine compounds, anti-folate compounds, thymidylate synthase (TS) inhibitors, anti-metabolite compounds, and pharmaceutically acceptable salts and solvates thereof.

40. A method according to claim 33, wherein the further chemotherapeutic agent is selected from the group of: 5-fluorouracil (FU), capecitabine, pemetrexed (MTA), pralatrexate (PDX), thymitaq (AG337), plevitrexed (ZD9331), BGC945, raltitrexed, GW1843, methotrexate (MTX), edatrexate (EDX), aminopterin (AMT), PT523, and neutrexin (trimetrexate), UFT and S-1 and pharmaceutically acceptable salts and solvates thereof.

41. A method according to claim 33, wherein the further chemotherapeutic agent is selected from the group of 5-fluorouracil, pralatrexate, capecitabine and pemetrexed.

42. A method according to claim 33 wherein the cancer is selected from the group of: colorectal cancer, pancreatic cancer, esophagal cancer, gastric cancer, head and neck cancer, prostate cancer, non small cell lung cancer, non-Hodgkin lymphoma and breast cancer.

43. A method according to claim 33, wherein the HDACi is a hydroxamic acid based HDAC inhibitor.

44. A method according to claim 33, wherein the HDACi is selected from PXD-101 (belinostat), vorinostat, panobinostat (hydroxamate), romidepsin (depsipeptide), SNDX-275, MGCD-0103, PCI24781, CHR-3996, ITF2357, SB939, JNJ26481585, JNJ16241199, valproic acid, and pharmaceutically acceptable salts and solvates thereof.

45. A method according to claim 33, wherein the subject shows a decrease in expression of TS, a decrease in expression of DPD, and an increase in expression of p21.
